(19) United States
(12) Patent Application Publication
(10) Pub. No.: US 2009/0169639 A1
(43) Pub. Date: Jul. 2, 2009

(54) PARTICLES FOR INJECTION AND PROCESSES FOR FORMING THE SAME

(75) Inventors: Goldi Kaul, Nyack, NY (US); Robert E. Richard, Wrentham, MA (US)

Correspondence Address: MAYER & WILLIAMS PC, 251 NORTH AVENUE WEST, 2ND FLOOR, WESTFIELD, NJ, 07090, US

(73) Assignee: BOSTON SCIENTIFIC SCIMED, INC., Maple Grove, MN (US)

(21) Appl. No.: 12/339,357

(22) Filed: Dec. 19, 2008

Related U.S. Application Data
(60) Provisional application No. 61/009,519, filed on Dec. 28, 2007.

(51) Int. Cl. A61K 9/14; A61K 47/30

(52) U.S. Cl. 424/499; 514/772.3

(57) ABSTRACT

In accordance with one aspect of the invention, injectable particles are provided which comprise (a) a vinyl formal polymer, (b) a glucosamine polymer, and (c) an ionically or covalently bound agent selected from therapeutic agents, cell surface binding agents, and combinations thereof. Other aspects of the invention pertain to methods of making and using such particles.

### STATEMENT OF RELATED APPLICATIONS

This application claims the benefit of U.S. Provisional Patent Application Ser. No. 61/009,519, filed Dec. 28, 2007, entitled “Particles For Injection And Processes For Forming The Same”, which is incorporated by reference herein in its entirety.

### FIELD OF THE INVENTION

The invention relates to particles for injection, to processes for forming the same, and to methods of using the same.

### BACKGROUND OF THE INVENTION

Injectable particles are employed in various capacities in the medical field. For example, many clinical situations benefit from regulation of the vascular, lymphatic or duct systems by restricting the flow of body fluid or secretions. For instance, the technique of embolization involves the therapeutic introduction of particles into the circulation to occlude blood vessels. Permanent or temporary occlusion of blood vessels is desirable for managing various diseases, disorders and conditions. For example, permanent or temporary occlusion of blood vessels can be used to either arrest or prevent hemorrhaging or to cut off blood flow to a structure or organ.

Various polymer-based injectable microspheres are currently employed to embolize blood vessels. These microspheres are usually introduced to the location of the intended embolization through microcatheters. Current commercially available embolic microspheres are composed of biostable polymers. Materials commonly used commercially for this purpose include polyvinyl alcohol (PVA), acetalized PVA (e.g., Contour SE™ embolic agent, Boston Scientific, Natick, Mass., USA) and crosslinked acrylic hydrogels (e.g., Embospheres®, Biosphere Medical, Rockland, Mass., USA). Similar devices have been used in chemoembolization to increase the residence time of a therapeutic agent after delivery. In one specific instance, a therapeutic agent (doxorubicin) has been directly added to hydrogel microspheres (prepared from N-acrylamidoacetaldehyde derivatized polyvinyl alcohol copolymerized with 2-acrylamido-2-methylpropane sulfonate) such that it can be released locally after delivery (e.g., DC Bead™ drug delivery chemoembolization system, Biocompatibles International plc, Farnham, Surrey, UK). Other examples of commercially available microspheres include glass microspheres with entrapped radioisotopes (e.g., ⁹⁰Y), in particular, TheraSpheres™, MDS Nordion, Ottowa, Canada and polymer microspheres that contain monomers that are capable of chelating radioisotopes (⁹⁰Y), in particular, SIR-Spheres®, SIRTex Medical, New South Wales, Australia.

It is also known to use polymer-based microspheres as augmentative materials for aesthetic improvement, including improvement of skin contour. Furthermore, polymer-based microspheres have been used as augmentative materials in the treatment of various diseases, disorders and conditions, including urinary incontinence, vesicourethral reflux, fecal incontinence, intrinsic sphincter deficiency (ISD) and gastro-esophageal reflux disease. For instance, a common method for treating patients with urinary incontinence is via periurethral or transperineal injection of a bulking agent that contains polymer-based microspheres. The bulking agent is injected into a plurality of locations, assisted by visual aids, causing the urethral lining to coapt.

### SUMMARY OF THE INVENTION

In accordance with one aspect of the invention, injectable particles are provided which comprise (a) a vinyl formal polymer, (b) a glucosamine polymer, and (c) an ionically or covalently bound agent selected from a therapeutic agent, a cell surface binding agent, and combinations thereof.

Other aspects of the invention pertain to methods of making such particles, to kits that comprise such particles, and to methods of treatment that employ such injectable particles.

These and various additional aspects, as well as various embodiments and advantages of the present invention will become immediately apparent to those of ordinary skill in the art upon review of the Detailed Description and any appended claims to follow.

### DETAILED DESCRIPTION

In accordance with one aspect of the invention, injectable particles are provided which comprise a vinyl formal polymer and a glucosamine polymer. The particles also comprise one or more ionically or covalently bound agent selected from a therapeutic agent, a cell surface binding agent, and combinations thereof.

The injectable particles may be used to treat various diseases and conditions in a variety of subjects. Subjects include vertebrate subjects, particularly humans and various warm-blooded animals, including pets and livestock. As used herein, “treatment” refers to the prevention of a disease or condition, the reduction or elimination of symptoms associated with a disease or condition, or the substantial or complete elimination of a disease or condition.

The injectable particles of the invention may vary in shape. In certain embodiments, they are substantially spherical, for example, having the form of a perfect (to the eye) sphere or the form of a near-perfect sphere such as a prolate spheroid (a slightly elongated sphere) or an oblate spheroid (a slightly flattened sphere), among other possibilities. In embodiments where the particles are substantially spherical, at least half of the particles (50% or more, for example, from 50% to 75% to 90% to 95% or more of a particle sample) may have a sphericity of 0.8 or more (e.g., from 0.80 to 0.85 to 0.9 to 0.95 to 0.97 or more). The sphericity of a collection of particles can be determined, for example, using a Beckman Coulter RapidVUE Image Analyzer version 2.06 (Beckman Coulter, Miami, Fla.). Briefly, the RapidVUE takes an image of continuous-tone (gray-scale) form and converts it to a digital form through the process of sampling and quantization. The system software identifies and measures the particles in an image. The sphericity of a particle, which is computed as Da/Dp (where Da=√(4A/π); Dp=P/π; A=pixel area; P=pixel perimeter), is a value from zero to one, with one representing a perfect circle.

The injectable particles of the invention can vary in size, with typical maximum dimensions (e.g., for a sphere, the diameter) ranging, for example, from 0.1 to 0.25 to 0.5 to 1 to 2.5 to 5 to 10 to 25 to 50 to 100 to 250 to 500 to 1000 to 2500 to 5000 microns (μm). For example, particles on the lower end of this range (e.g., ranging from 0.1 to 5 microns) may be used for localized drug or gene delivery (e.g., in the treatment of solid tumors), whereas particles on the upper end of this range (e.g., ranging from 100 to 5000 microns) may be used for embolic or tissue bulking applications, among other possibilities.

For a collection of particles, the arithmetic mean maximum dimension for the group typically ranges, for example, from 0.1 to 0.25 to 0.5 to 1 to 2.5 to 5 to 10 to 25 to 50 to 100 to 250 to 500 to 1000 to 2500 to 5000 microns (μm). The arithmetic mean maximum dimension of a group of particles can be determined using a Beckman Coulter RapidVUE Image Analyzer version 2.06 (Beckman Coulter, Miami, Fla.), described above. The arithmetic mean maximum dimension of a group of particles (e.g., in a composition) can be determined by dividing the sum of the maximum dimensions (which, for a sphere, is the diameter) of all of the particles in the group by the number of particles in the group.

As used herein a “polymeric particle” is one that contains polymers, for example, from 25 wt % or less to 50 wt % to 75 wt % to 90 wt % to 95 wt % to 97.5 wt % to 99 wt % or more polymers.

As used herein, “polymers” are molecules that contain multiple copies of one or more types of constitutional units, commonly referred to as monomers. The number of monomers/constitutional units within a given polymer may vary widely, ranging, for example, from 5 to 10 to 25 to 50 to 100 to 1000 to 10,000 or more constitutional units. As used herein, the term “monomers” may refer to free monomers and to those that are incorporated into polymers, with the distinction being clear from the context in which the term is used.

Polymers for use in the present invention may have a variety of architectures, including cyclic, linear and branched architectures. Branched architectures include star-shaped architectures (e.g., architectures in which three or more chains emanate from a single branch point), comb architectures (e.g., architectures having a main chain and a plurality of side chains, such as graft polymers), dendritic architectures (e.g., arborescent and hyperbranched polymers), and networked architectures (e.g., crosslinked polymers), among others.

Polymers containing a single type of monomer are referred to herein as homopolymers, whereas polymers containing two or more types of monomers are referred to herein as copolymers. The two or more types of monomers within a given copolymer may be present in any of a variety of distributions including random, statistical, gradient and periodic (e.g., alternating) distributions, among others. One particular type of copolymer is a “block copolymer,” which is a copolymer that contains two or more polymer blocks of different composition. As used herein, a “block” or “polymer block” is a grouping of constitutional units (e.g., 5 to 10 to 25 to 50 to 100 to 250 to 500 to 1000 or more units). Blocks can be unbranched or branched. Blocks can contain a single type of constitutional unit (also referred to herein as “homopolymeric blocks”) or multiple types of constitutional units (also referred to herein as “copolymeric blocks”) which may be present, for example, in a random, statistical, gradient, or periodic (e.g., alternating) distribution.

Polymeric particles in accordance with the invention may be biostable, bioresorbable, or partially biostable and partially bioresorbable.

As used herein, a polymeric particle is “bioresorbable” if it disintegrates in vivo due to one or more mechanisms such as dissolution, biodegradation, and so forth. On the other hand, a polymeric particle is “biostable” if it does not disintegrate in vivo.

As used herein, a polymer is “biodegradable” if it undergoes bond cleavage along the polymer backbone in vivo, regardless of the mechanism of bond cleavage (e.g., enzymatic breakdown, hydrolysis, oxidation, etc.).

In some embodiments of the invention, the polymeric particles are hydrogel particles. As used herein, a “hydrogel” is a crosslinked hydrophilic polymer (e.g., a polymer network) which swells when placed in water or biological fluids, but remains insoluble due to the presence of crosslinks, which may be, for example, physical, chemical, or both. For instance, a hydrogel particle in accordance with the invention may undergo swelling in water such that its longest linear cross-sectional dimension (e.g., for a sphere, the diameter) increases by 5% or less to 10% to 15% to 20% to 25% or more. In some instances, the insolubility of the hydrogel is not permanent, and the particles are ultimately bioresorbed.

As noted above, in accordance with one aspect of the invention, injectable particles are provided which comprise a vinyl formal polymer and a glucosamine polymer, as well as one or more optional supplemental polymers in some embodiments.

As used herein, vinyl formal polymers include homopolymers and copolymers, that contain vinyl formal monomers,

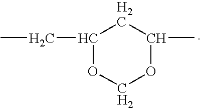

One way of forming vinyl formal polymer is via vinyl alcohol polymers. As used herein, vinyl alcohol polymers include homopolymers and copolymers that contain vinyl alcohol monomers,

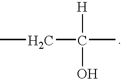

Vinyl alcohol, H₂C═CHOH, does not exist in a stable free form, due to keto-enol rearrangement with its tautomer (acetaldehyde). Thus, vinyl alcohol polymers are typically produced from vinyl ester polymers including vinyl acetate polymers. As used herein, vinyl acetate polymers include homopolymers and copolymers that contain vinyl acetate monomers,

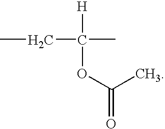

The vinyl ester polymers are subjected to hydrolysis to convert the ester groups to hydroxyl groups, forming vinyl alcohol monomers. The hydrolysis reaction, however, does not typically go to completion, resulting in polymers with a certain degree of hydrolysis that depends on the extent of reaction. Thus, vinyl alcohol polymers commonly include both vinyl alcohol monomers and vinyl ester monomers, typically, vinyl acetate monomers.

Commercial polyvinyl alcohol grades are available with varying degrees of hydrolysis (e.g., 50% to 99% or more) including grades with high degrees of hydrolysis (above 98.5%). The degree of hydrolysis (or, conversely, the ester group content) of the polymer has an effect on its chemical properties, crystallizability, and solubility, among other properties. For example, degrees of hydrolysis and polymerization are known to affect the solubility of PVA in water, with PVA grades having high degrees of hydrolysis being known to have reduced solubility in water relative to those having low degrees of hydrolysis. For further information on PVA (as well as PVA hydrogels), see, e.g., C. M. Hassan et al., “Structure and Applications of Poly(vinyl alcohol) Hydrogels Produced by Conventional Crosslinking or by Freezing/Thawing Methods,” Adv. Polym. Sci., 153, 37-65 (2000) and N. A. Peppas et al., “Hydrogels in Biology and Medicine: From Fundamentals to Bionanotechnology”, Adv. Mater., 18, 1345-1360 (2006).

As noted in Pub. No. US 2003/0185895 to Lanphere et al., it is possible under certain circumstances to react a vinyl alcohol polymer with an aldehyde (formaldehyde) in the presence of an acid such that the reaction is primarily a 1,3 acetalization reaction,

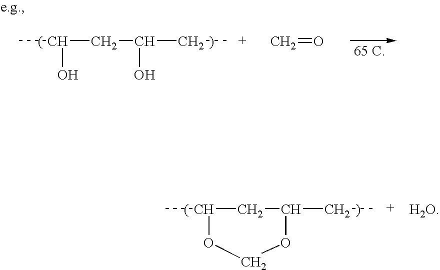

Such intra-chain acetalization reaction can be carried out with relatively low probability of inter-chain crosslinking. Since the reaction proceeds in a random fashion, there will generally be leftover —OH groups that do not react with adjacent groups. Moreover, the residual vinyl ester groups do not take part in the above reactions. Thus, vinyl alcohol polymers crosslinked in this fashion are commonly vinyl formal copolymers that contain the following: vinyl formal monomers, vinyl alcohol monomers and vinyl ester monomers, typically vinyl acetate monomers. Examples of ranges for each of these monomer units are as follows, among others: 40 to 90 mole % vinyl formal monomers, 5-60 mole % vinyl alcohol monomers, and 0 to 20 mole % vinyl ester monomers. The weight percent of a monomer unit in a polymer can be measured using solid-state NMR spectroscopy.

As previously indicated, in addition to a vinyl formal polymer, particles in accordance with the invention also include a glucosamine polymer. As used herein, glucosamine polymers include homopolymers and copolymers that contain glucosamine monomers, for example, D-glucosamine monomers,

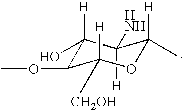

In certain preferred embodiments of the invention, the glucosamine polymer is a copolymer of glucosamine and N-acetyl-glucosamine. A specific example of such a polymer is chitosan. Chitosan is a modified polysaccharide containing randomly distributed β-(1-4)-linked D-glucosamine and N-acetyl-D-glucosamine monomer units. Chitosan is produced commercially by the alkaline N-deacetylation of chitin, which is a cellulose-like polymer consisting primarily of unbranched chains of modified glucose, specifically N-acetyl-D-glucosamine,

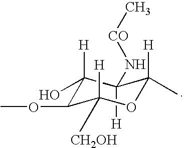

The degree of deacetylation in commercial chitosans generally ranges from 60 to 70 to 80 to 90 to 100% although essentially any degree of deacetylation is possible. Chitosan is positively charged in acidic to neutral solutions with a charge density that is dependent on the pH and the degree of deacetylation. The pka value of chitosan generally ranges from 6.1 to 7.0, depending on the degree of deacetylation. Thus, while typically substantially insoluble in distilled water, chitosan is generally soluble in dilute aqueous acidic solutions (e.g., pH ˜6.5 or less).

In some embodiments, hydrophilic moieties (e.g., saccharide moieties, etc.) may be attached to a portion of the glucosamine monomers within the glucosamine polymers of the invention via a suitable coupling methodology. For example, galactose moieties, specifically, residues of lactobionic acid (i.e., 4-(β-D-galactosido)-D-gluconic acid),

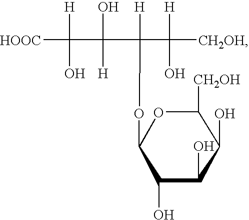

may be linked at some of the primary amino groups of chitosan (e.g., at a substitution degree varying from 8 to 23%) using activated succinimide esters in the presence of a water-soluble carbodiimide to form galactosylated chitosan, as described in A. V. Il'ina et al., Applied Biochemistry and Microbiology, 2007, 43(1), 73-77, among other possibilities. Such moieties may be attached to the glucosamine polymers either before or after injectable particles are formed from the glucosamine polymers. Hydrophilic saccharide moieties such as galactose moieties are desirable in that they render the polymer more soluble at neutral pH (thus improving the manipulability of the polymer during processing). Galactose moieties are also desirable and in that they can act as targeting moieties for cell surfaces.

In addition to a vinyl formal polymer and a glucosamine polymer, the particles of the invention may also optionally contain one or more supplemental polymers, which may be hydrophobic, hydrophilic or amphiphilic, which may be charged or uncharged at neutral pH, and which may be biostable or biodegradable. Specific supplemental polymers may be selected, for example, from one or more suitable members of the following, among others: polycarboxylic acid homopolymers and copolymers including polyacrylic acid, polymethacrylic acid, ethylene-methacrylic acid copolymers and ethylene-acrylic acid copolymers, where some of the acid groups can be neutralized with either zinc or sodium ions (commonly known as ionomers); acetal homopolymers and copolymers; acrylate and methacrylate homopolymers and copolymers (e.g., n-butyl methacrylate); cellulosic homopolymers and copolymers, including cellulose acetates, cellulose nitrates, cellulose propionates, cellulose acetate butyrates, cellophanes, rayons, rayon triacetates, and cellulose ethers such as carboxymethyl celluloses and hydroxyalkyl celluloses; polyoxymethylene homopolymers and copolymers; polyimide homopolymers and copolymers such as polyether block imides, polyamidimides, polyesterimides, and polyetherimides; polysulfone homopolymers and copolymers including polyarylsulfones and polyethersulfones; polyamide homopolymers and copolymers including nylon 6,6, nylon 12, polycaprolactams, polyacrylamides and polyether block amides; resins including alkyd resins, phenolic resins, urea resins, melamine resins, epoxy resins, allyl resins and epoxide resins; polycarbonate homopolymers and copolymers; polyacrylonitrile homopolymers and copolymers; polyvinylpyrrolidone homopolymers and copolymers (cross-linked and otherwise); homopolymers and copolymers of vinyl monomers including polyvinyl halides such as polyvinyl chlorides, ethylene-vinyl acetate copolymers (EVA), polyvinylidene chlorides, polyvinyl ethers such as polyvinyl methyl ethers, polystyrenes, styrene-maleic anhydride copolymers, vinyl-aromatic-alkylene copolymers, including styrene-butadiene copolymers, styrene-ethylene-butylene copolymers (e.g., a polystyrene-polyethylene/butylene-polystyrene (SEBS) copolymer, available as Kraton® G series polymers), styrene-isoprene copolymers (e.g., polystyrene-polyisoprene-polystyrene), acrylonitrile-styrene copolymers, acrylonitrile-butadiene-styrene copolymers, styrene-butadiene copolymers and styrene-isobutylene copolymers (e.g., polyisobutylene-polystyrene and polystyrene-polyisobutylene-polystyrene (SIBS) block copolymers such as those disclosed in U.S. Pat. No. 6,545,097 to Pinchuk), poly[(styrene-co-p-methylstyrene)-b-isobutylene-b-(styrene-co-p-methylstyrene)] (SMIMS) triblock copolymers described in S. J. Taylor et al., Polymer 45 (2004) 4719-4730; polyphosphonate homopolymers and copolymers; polysulfonate homopolymers and copolymers, for example, sulfonated vinyl aromatic polymers and copolymers, including block copolymers having one or more sulfonated poly(vinyl aromatic) blocks and one or more polyalkene blocks, for example, sulfonated polystyrene-polyolefin-polystyrene triblock copolymers such as the sulfonated SEBS copolymers described in U.S. Pat. No. 5,840,387, and sulfonated versions of SIBS and SMIMS, which polymers may be sulfonated, for example, using the processes described in U.S. Pat. No. 5,840,387 and U.S. Pat. No. 5,468,574, among other sulfonated block copolymers; polyvinyl ketones, polyvinylcarbazoles, and polyvinyl esters such as polyvinyl acetates; polybenzimidazoles; polyalkyl oxide homopolymers and copolymers including polyethylene oxides (PEO); polyesters including polyethylene terephthalates and aliphatic polyesters such as homopolymers and copolymers of lactide (which includes lactic acid as well as d-, l- and meso lactide), epsilon-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, and 6,6-dimethyl-1,4-dioxan-2-one (a copolymer of poly(lactic acid) and poly(caprolactone) is one specific example); polyether homopolymers and copolymers including polyarylethers such as polyphenylene ethers, polyether ketones, polyether ether ketones; polyphenylene sulfides; polyisocyanates; polyolefin homopolymers and copolymers, including polyalkylenes such as polypropylenes, polyethylenes (low and high density, low and high molecular weight), polybutylenes (such as polybut-1-ene and polyisobutylene), polyolefin elastomers (e.g., santoprene), ethylene propylene diene monomer (EPDM) rubbers, poly-4-methyl-pen-1-enes, ethylene-alpha-olefin copolymers, ethylene-methyl methacrylate copolymers and ethylene-vinyl acetate copolymers; fluorinated homopolymers and copolymers, including polytetrafluoroethylenes (PTFE), poly(tetrafluoroethylene-co-hexafluoropropene) (FEP), modified ethylene-tetrafluoroethylene copolymers (ETFE), and polyvinylidene fluorides (PVDF); silicone homopolymers and copolymers; thermoplastic polyurethanes (TPU); elastomers such as elastomeric polyurethanes and polyurethane copolymers (including block and random copolymers that are polyether based, polyester based, polycarbonate based, aliphatic based, aromatic based and mixtures thereof, p-xylylene polymers; polyiminocarbonates; copoly(ether-esters) such as polyethylene oxide-polylactic acid copolymers; polyphosphazines; polyalkylene oxalates; polyoxaamides and polyoxaesters (including those containing amines and/or amido groups); polyorthoesters; polyamine and polyimine homopolymers and copolymers; biopolymers, for example, polypeptides including anionic polypeptides such as polyglutamate and cationic polypeptides such as polylysine, proteins, polysaccharides, and fatty acids (and esters thereof), including fibrin, fibrinogen, collagen, elastin, gelatin, starch, glycosaminoglycans such as hyaluronic acid; as well as further copolymers, derivatives (e.g., esters, etc.) and mixtures of the foregoing.

As previously noted, particles in accordance with the invention also contain one or more agents selected from therapeutic agents, cell surface binding agents or both. Among other characteristics, such agents may be, for example, hydrophobic, hydrophilic or amphiphilic, and they may be negatively charged, positively charged, zwitterionic, or uncharged at neutral pH. Such agents may be bound to the particles of the invention, for example, by covalent bonding or by ion-based electrostatic interactions.

Cell surface binding agents may be employed in the particles of the invention in order to bind the particles at a desired location with in the vasculature. Depending on the specificity, such binding agents may help the particles find their ideal location in the body. By targeting specific tissues, the particles can exert a local effect (e.g., a local chemotherapeutic effect), potentially reducing particle migration to non-intended regions of the body due to blood flow and thus reducing toxic effects on healthy cells that are remote from the treatment region.

Examples of cell surface binding agents include lectins, cell adhesion molecules (CAMs), extracellular matrix components, peptide binding sequences, and antibodies for particular cell surface components, among others.

Lectins are sugar-binding proteins and glycoprotein receptors of non-immune origin that are capable of binding specific sugars (e.g., those associated with polysaccharides, glycoproteins, glycolipids, etc.) including those associated with cell surfaces. For example, in certain embodiments of the invention, lectins are selected which bind to particular cancer cells. The rationale behind lectin-mediated drug targeting is that most cell surface proteins and many lipids in cell membranes are glycosylated and that these glycans are binding sites for lectins. The combination of a small number of sugars can produce a vast range of different chemical structures. Different cell types express different glycan arrays and, in particular, diseased cells such as transformed or cancerous cells often express different glycans compared with their normal counterparts. Therefore, lectins may be used as carrier molecules to target drugs specifically to different cells and tissues. For example, E. coli K99 fimbriae have been used to target the corticosteroid 6-methyl-prednisolone to the affected part of the GI tract of patients with Crohn's disease. See, e.g., A. Bernkop-Schnürch et al., “An adhesive drug delivery system based on K99-fimbriae,” Eur. J. Pharm. Sci. 3 (1995), pp. 293-299 and A. Bernkop-Schnürch et al., “Bacterial adhesins as a drug carrier: covalent attachment of K99 fimbriae to 6-methylprednisolone,” Pharmazie 52 (1997), pp. 41-44. Scientists have been working to develop of drug-bearing polymers with lectins to target to diseased cells.

CAMs are molecules that are expressed on the surfaces of cells which mediate the adhesion of the cell to other cells or to the extracellular matrix. Examples of CAMS include Immunoglobulin superfamily CAMs, integrins, cadherins and selectins. For example, monoclonal antibodies directed against the L1 cell adhesion molecule were shown recently to inhibit growth of target tumor cells in vitro and the growth of tumor cells in vivo in nude mice. Ilse Novak-Hofer, Cancer Biotherapy & Radiopharmaceuticals, 2007, 22(2): 175-184.

Extracellular matrix components include structural proteins such as collagen and elastin, specialized proteins such as fibronectin, laminin and fibrillin, glycosaminoglycans such as hyaluronates, dermatan sulfates, chondroitin 4- and 6-sulfates, heparin and heparan sulfates, keratan sulfates, and extracellular matrix component subunits such as RGD containing peptides.

Peptide binding sequences for binding cell surfaces, including peptides containing one or more of the following sequences: RGD, YIGSR, and positively charged peptide sequences (due to the presence of basic amino acids such as lysine [K], arginine [R], and/or histidine [H]) for binding negatively charged sulfate and carboxylate groups of cell surface proteoglycans, examples of which include PRRARV (derived from fibronectin), PRRGRV (derived from fibronectin), YEKPGSPPREVVPRPRPGV (derived from fibronectin), RPSLAKKQRFRHRNRKGYRSQRGHSRGR (derived from vitronectin), RIQNLLKITNLRIKFVK (derived from laminin), and RYVVLPRPVCFEKGMNYTVR (derived from laminin). See, e.g., Stephen P. Massia et al., The Journal of Biological Chemistry, Vol. 267, No. 14, 1992, pp. 10133-10141 and the references cited therein for further discussion of these sequences.

Examples of antibodies for particular cell surface components include anti-VEGF antibodies and monoclonal antibodies. An anti-VEGF antibody binds VEGF (which tumors employ to support angiogenesis), and may also block the cell surface receptor from producing VEGF and promoting angiogenesis.

As previously noted, particles in accordance with the present invention comprise one or more ionically or covalently bound therapeutic agent in some embodiments. Advantages of binding the therapeutic agent(s) include reduction or prevention of premature drug leakage prior to arrival of the particles at the intended treatment site (e.g., a tumor, etc.)

Examples of therapeutic agents vary widely and include antioxidants; anti-angiogenic agents; calcium entry blockers (e.g., verapamil, diltiazem, nifedipine); steroidal and non-sterioidal anti-inflammatory agents (e.g., dexamethasone, prednisolone, corticosterone, budesonide, estrogen, acetyl salicylic acid, sulfasalazine, mesalamine, etc.); anesthetic agents (e.g., lidocaine, bupivacaine and ropivacaine); protein kinase and tyrosine kinase inhibitors; anti-proliferative agents; cytostatic agents (i.e., agents that prevent or delay cell division in proliferating cells, for example, by inhibiting replication of DNA or by inhibiting spindle fiber formation) (e.g., toxins, methotrexate, adriamycin, radionuclides, protein kinase inhibitors such as staurosporin and diindoloalkaloids, etc.), agents that inhibit intracellular increase in cell volume (i.e., the tissue volume occupied by a cell) such as cytoskeletal inhibitors (e.g., colchicine, vinblastin, cytochalasins, paclitaxel, etc.) or metabolic inhibitors (e.g., staurosporin, Pseudomonas exotoxin, modified diphtheria and ricin toxins, etc.); trichothecenes (e.g., a verrucarin or roridins); agents acting as an inhibitor that blocks cellular protein synthesis and/or secretion or organization of extracellular matrix (i.e., an “anti-matrix agent” such as colchicine or tamoxifen); various pharmaceutically acceptable salts and derivatives of the foregoing, and combinations of the foregoing, among other agents.

Examples of therapeutic agents which may be used in the particles of the invention thus include toxins (e.g., ricin toxin, radioisotopes, etc.) and other agents able to kill undesirable cells (e.g., those making up cancers and other tumors such as uterine fibroids) or to slow or arrest growth of undesirable cells, among other agents.

Further specific examples of therapeutic agents for use in the particles of the invention, not necessarily exclusive of those above, may be selected from suitable members of the following: radioisotopes (e.g, ⁹⁰Y, ³²P, ¹⁸F, ¹⁴⁰La, ¹⁵³Sm, ¹⁶⁵Dy, ¹⁶⁶Ho, ¹⁶⁹Er, ¹⁶⁹Yb, ¹⁷⁷Lu, ¹⁸⁶Re, ¹⁸⁸Re, ¹⁰³Pd, ¹⁹⁸Au, ¹⁹²Ir, ⁹⁰Sr, ¹¹¹In or ⁶⁷Ga), which may be covalently bound or non-covalently bound to another species, antineoplastic/antiproliferative/anti-mitotic agents including antimetabolites such as folic acid analogs/antagonists (e.g., methotrexate, etc.), purine analogs (e.g., 6-mercaptopurine, thioguanine, cladribine, which is a chlorinated purine nucleoside analog, etc.) and pyrimidine analogs (e.g., cytarabine, fluorouracil, etc.), alkaloids including taxanes (e.g., paclitaxel, docetaxel, etc.), alkylating agents such as alkyl sulfonates, nitrogen mustards (e.g., cyclophosphamide, ifosfamide, etc.), nitrosoureas, ethylenimines and methylmelamines, other aklyating agents (e.g., dacarbazine, etc.), antibiotics and analogs (e.g., daunorubicin, doxorubicin, idarubicin, mitomycin, bleomycins, plicamycin, etc.), antiestrogens (e.g., tamoxifen), antiandrogens (e.g., flutamide), platinum complexes (e.g., cisplatin, carboplatin, etc.), antineoplastic enzymes (e.g., asparaginase, etc.), agents affecting microtubule dynamics (e.g., vinblastine, vincristine, colchicine, Epo D, epothilone), caspase activators, proteasome inhibitors, angiogenesis inhibitors (e.g., statins such as endostatin, cerivastatin and angiostatin, squalamine, etc.), olimus family drugs (e.g., sirolimus (rapamycin), everolimus, tacrolimus, zotarolimus, etc.), etoposides, as well as many others (e.g., hydroxyurea, flavopiridol, procarbizine, mitoxantrone, campothecin, etc.), various pharmaceutically acceptable salts and derivatives (e.g., esters, etc.) of the foregoing, and combinations of the foregoing, among other agents.

For tissue bulking applications (e.g., urethral bulking, cosmetic bulking, etc.), specific beneficial therapeutic agents include those that promote collagen production, including proinflammatory agents and sclerosing agents such as those listed Pub. No. US 2006/0251697.

Various procedures have associated with them some degree of pain. Thus, in certain embodiments, the injectable particles of the invention contain one or more agents selected from narcotic analgesics, non-narcotic analgesics, local anesthetic agents and other pain management agents.

As previously indicated, therapeutic agents and/or cell surface binding agents such as those described above, among others, may be bound to the particles of the present invention via covalent binding or ion-based electrostatic interactions (e.g., charge-charge interactions or charge-dipole interactions, such as those associated with ion exchange, complexation, coordination, chelation, etc.).

As also previously indicated, particles in accordance with the present invention comprise a vinyl formal polymer (e.g., formalized polyvinyl alcohol, etc.) and a glucosamine polymer (e.g., chitosan, etc.).

Where formed from vinyl alcohol polymers, vinyl formal polymers will typically contain residual hydroxyl groups in an amount depending upon the degree to which the hydroxyl groups are converted into vinyl formal groups. Thus, in certain embodiments of the invention, vinyl formal polymers may be employed which have a lower degree of formalization, in order to increase the mol % of hydroxyl groups within the polymer. Glucosamine polymers such as chitosan also contain hydroxyl groups and amine groups as well. Such hydroxyl and amine groups are relatively reactive and can be used to covalently bind the therapeutic agents and/or cell surface binding agents to these polymers, and thus to the particles of the invention. Such binding may be conducted either before or after injectable particles are formed from the vinyl formal and glucosamine polymers.

For example, in some embodiments, sulfonyl chloride or one of its derivatives can be reacted with hydroxyl groups in the above polymers to form sulfonyl esters, which can in turn react with amino groups such as those found in various therapeutic agents and/or cell surface binding agents (e.g., small molecule and polymeric agents having amino groups, including poly(amino acid) containing agents such as peptides, proteins, glycoproteins, lipoproteins, etc.) Stephen P. Massia et al., The Journal of Biological Chemistry, Vol. 267, No. 14, 1992, pp. 10133-10141 describe a process whereby glycophase glass is activated with a sulfonyl chloride deriviative (i.e., 2,2,2-trifluoroethanesulfonyl chloride) to form surface-immobilized sulfonyl esters. Various amines (e.g., monoamines, diamines, thioamines, and amino acids) were prepared in a coupling buffer and coupled to the sufonyl-ester-activated glass surface. Analogous procedures may be used in accordance with the invention to react sulfonyl chloride or a derivative thereof with hydroxyl groups on the vinyl formal and glucosamine polymers, thereby forming sulfonyl esters which can be subsequently used to couple various agents via reaction with the amino and/or thiol groups of such agents.

As another example, in some embodiments, the amino groups of the chitosan are used to participate in carbodiimide coupling with carboxyl groups such as those found in various therapeutic agents and/or cell surface binding agents (e.g., small molecule and polymeric agents having carboxyl groups, including poly(amino acid) containing agents such as peptides, proteins, glycoproteins, lipoproteins, etc.). Carbodiimide couple agents are well known and include, for example, 1-alkyl-3-(3-dimethylaminopropyl)carbodiimides such as 1-ethyl-3-(3-dimethylamninopropyl)carbodiimide hydrochloride (EDC), 1-benzyl-3-(3-dimethylaminopropyl)carbodiimide (BDC), 1-cyclohexyl-3-(2-morpholinyl-4-ethyl)carbodiimide (CMC), and 1,3-dicyclohexylcarbodiimide (DCC). In some instances, supplemental agents may optionally be added to enhance the coupling reaction, including, for example, N-succinimide or N-hydroxysuccinimide, among others.

Using the above and other techniques, various agents (e.g., therapeutic agents and/or cell surface binding agents, etc.) may be covalently linked to one or more particle forming polymers (e.g., the vinyl formal polymer, the glucosamine polymer, and any optional supplemental polymer) either before or after injectable particles are formed from such polymers.

In other embodiments, charged therapeutic agents and/or cell surface binding agents are ionically bound to charged polymers within the particles of the invention.

Examples of positively charged therapeutic agents and cell surface binding agents include small molecule and polymeric agents having positively charged groups (e.g., having one or more —NH₃⁺ groups, ═NH₂⁺ groups, ≡NH⁺ groups, ═N⁺═ groups, etc.), including poly(amino acid)-containing agents such as peptides, proteins, glycoproteins and lipoproteins, particularly those having a preponderance of lysine (isoelectric point of pH ˜9.8), arginine (isoelectric point of pH ˜10.8) and/or histidine (isoelectric point of pH ˜7.5).

Examples of negatively charged therapeutic agents and cell surface binding agents include small molecule and polymeric agents having negatively charged groups (e.g.,

—COO⁻, —SO₃⁻, —OSO₃⁻, —PO₂(OH)⁻, —PO₃²⁻, —OPO₂(OH)⁻, —OPO₃²⁻ groups, etc.) including poly(amino acid)-containing agents such as peptides, proteins, glycoproteins and lipoproteins, particularly those having a preponderance of glutamic acid (isoelectric point of pH ˜3.2) and/or or aspartic acid (isoelectric point of pH ˜2.8).

Examples of negatively charged cell surface binding agents include fibronectin, sialic acid and albumin. Examples of negatively charged therapeutic agents include methotrexate, ketorolac and bromopyruvic acid. Examples of positively charged therapeutic agents include radioisotopes, doxorubicin, campothecin and irinotecan.

With regard to charged polymers for use within the particles of the invention, it is noted that the pka value of the amino group of the glucosamine units within glucosamine polymers is about 6.5, such that these glucosamine polymers are generally positively charged and soluble in dilute aqueous acidic solutions (e.g., pH ˜6.5 or less). Accordingly, therapeutic agents and/or cell surface binding agents which are negatively charged (or which have negatively charged domains) in such solutions may be electrostatically bound to the glucosamine polymers.

In other embodiments, polymers within the particles of the invention are positively or negatively charged at neutral pH. For instance, in some embodiments, positively charged polymers or negatively charged polymers are covalently coupled to the vinyl formal polymer and/or the glucosamine polymer found within the particles. For example, positively or negatively charged polymers containing amino groups (e.g., polyamino acids, which contain at least one terminal amino group, and commonly contain one or more side amino groups) may be covalently bound to sulfonyl-chloride-derivatized vinyl formal polymers and/or sulfonyl-chloride-derivatized glucosamine polymers, as described above. As another example, positively or negatively charged polymers containing carboxyl groups (e.g., polyamino acids, which contain at least one terminal carboxyl group, and commonly one or more side carboxyl groups) may be covalently bound to the amine groups of glucosamine polymers via carbodiimide coupling, as described above. Examples of positively charged poly(amino acids) include those containing a preponderance of lysine, arginine and/or histidine, including poly-1-lysine, poly-1-arginine and poly-1-histidine, among many others. Examples of negatively charged poly(amino acids) include those containing a preponderance of glutamic acid and/or or aspartic acid, including poly-1-glutamic acid, and poly-1-aspartic acid, among many others.

Such charged polymers may be covalently linked to one or more particle forming polymers (i.e., the vinyl formal polymer, the glucosamine polymer, and any optional supplemental polymer) either before or after injectable particles are formed from the particle forming polymers.

In other embodiments, supplemental polymers that are positively or negatively charged at neutral pH are blended with the vinyl formal polymer, the glucosamine polymer and any further optional supplemental polymer.

Positively charged polymers may be selected, for instance, from suitable members of the following: polyamines, including polyamidoamines, poly(amino methacrylates) including poly(dialkylaminoalkyl methacrylates) such as poly(dimethylaminoethyl methacrylate) and poly(diethylaminoethyl methacrylate), polyvinylamines, polyvinylpyridines including quaternary polyvinylpyridines such as poly(N-ethyl-4-vinylpyridine), poly(vinylbenzyltrimethylamines), polyallylamines such as poly(allylamine hydrochloride) (PAH) and poly(diallyldialklylamines) such as poly(diallyldimethylammonium chloride), polyimines including polyalkyleneimines such as polyethyleneimines, polypropyleneimines and ethoxylated polyethyleneimines, polycationic peptides and proteins, and polycationic polysaccharides such as cationic starch, as well as copolymers, derivatives and combinations of the preceding, among various others.

Negatively charged polymers may be selected, for instance, from suitable members of the following: polysulfonates such as polyvinylsulfonates, poly(styrenesulfonates) such as poly(sodium styrenesulfonate) (PSS), sulfonated poly(tetrafluoroethylene), sulfonated polymers such as those described in U.S. Pat. No. 5,840,387, including sulfonated styrene-ethylene/butylene-styrene triblock copolymers, sulfonated styrenic homopolymers and copolymers such as a sulfonated versions of the polystyrene-polyolefin copolymers described in U.S. Pat. No. 6,545,097 to Pinchuk et al., which polymers may be sulfonated, for example, using the processes described in U.S. Pat. No. 5,840,387 and U.S. Pat. No. 5,468,574, as well as sulfonated versions of various other homopolymers and copolymers, polysulfates such as polyvinylsulfates, polycarboxylates such as acrylic acid polymers and salts thereof (e.g., ammonium, potassium, sodium, etc.), for instance, those available from Atofina and Polysciences Inc., methacrylic acid polymers and salts thereof (e.g., EUDRAGIT, a methacrylic acid and ethyl acrylate copolymer), carboxymethylcellulose, carboxymethylamylose and carboxylic acid derivatives of various other polymers, polyanionic peptides and proteins, polymers and copolymers of uronic acids such as mannuronic acid, galatcuronic acid and guluronic acid, polyphosphates such as phosphoric acid derivatives of various polymers, polyphosphonates such as polyvinylphosphonates, as well as copolymers, derivatives and combinations of the preceding, among various others.

In addition to therapeutic agents and/or cell surface binding agents, injectable particles in accordance with the invention may also be optionally provided with various optional supplemental agents, including imaging contrast agents such as radiopaque agents, agents that are visible under magnetic resonance imaging (MRI-visible materials), ferromagnetic agents and/or ultrasound contrast agents, among others. These agents can, for example, be covalently bonded to or non-covalently associated with the particles. Various radiopaque materials, MRI-visible materials, ferromagnetic materials, and contrast agents are described, for example, in Pub. No. US 2004/0101564 A1 to Rioux et al.

Polymeric particles for use in the invention may be formed by any suitable particle forming method, including emulsion/solvent evaporation methods, precipitation methods, and droplet solidification methods, among many others.

In certain embodiments, the particles of the invention are stabilized via covalent crosslinking, non-covalent crosslinking, or both.

Polyols such as PVA, vinyl formal polymers (which typically contain residual hydroxyl groups, particularly low-formalized PVA) and glucosamine polymers (which contain hydroxyl groups on the ring of the glucosamine monomer) can be covalently crosslinked through the use of chemical crosslinking agents. Some of the common chemical crosslinking agents that may be used include dialdehydes such as glutaraldehyde and succindialdehyde, and monoaldehydes such as acetaldehyde and formaldehyde. In the presence of an acid (e.g., sulfuric acid, acetic acid, etc.) these crosslinking agents form acetal bridges between the pendant hydroxyl groups found on the polyol chains. For example, acetal formation may link two alcohol moieties together according to the following scheme:

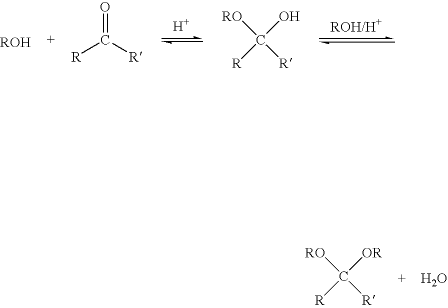

where R and R′ are organic groups. For species with multiple hydroxyl groups, including polyols such as PVA, two hydroxyl groups within the same molecule may react according to the following scheme:

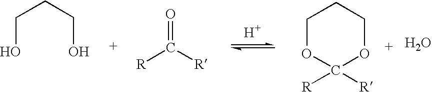

For example, as noted above, in certain instances, the reaction of PVA with an formaldehyde in the presence of an acid is primarily a 1,3 acetalization within individual chains.

In accordance with certain embodiments, particles may be formed by crosslinking a solution of a vinyl alcohol polymer and a glucosamine polymer with formaldehyde in the presence of an acid. As part of its role as a crosslinking agent, the formaldehyde creates polyvinyl formal from the PVA. In other embodiments, particles may be formed by crosslinking a solution of a vinyl formal polymer and a glucosamine polymer with an aldehyde (e.g., glutaraldehyde, succinaldehyde, acetaldehyde, formaldehyde, etc.) in the presence of an acid. In this regard, S. Gunasekaran et al., “Swelling of pH-Sensitive Chitosan-Poly(vinyl alcohol) Hydrogels,” Journal of Applied Polymer Science, Vol. 102, 4665-4671 (2006) describe a process in which glutaraldehyde is added to a solution of chitosan and PVA in dissolved in acetic acid, whereupon chitosan/PVA beads were formed by the slow dropping of the chitosan-PVA-glutaraldehyde solution into oleic acid.

In certain non-covalent crosslinking embodiments, charged polymers such as glucosamine polymers or various other charged polymers discussed herein can be can ionically crosslinked using suitable counterions.

For instance, negatively charged polymers can be crosslinked using multivalent species (e.g., Ca²⁺, Fe²⁺, Fe³⁺, etc.) or using polycationic species including low molecular weight polycationic counterions such as aliphatic or aromatic diamines or triamines, or using polymeric counterions, suitable members of which may be selected from the positively charged polymers set forth above.

On the other hand, positively charged polymers can be crosslinked using polyanionic species including low molecular weight polyanionic counterions, for example, aromatic or aliphatic diacids such as succinic acid, glutamic acid, aspartic acid, aromatic or aliphatic triacids such as citric acid, or other low molecular weight polycationic species such as tripolyphosphate, or polymeric counterions, suitable members of which may be selected from the negatively charged polymers set forth above.

As one specific example, particles in accordance with the present invention can be formed by dissolving chitosan in glacial acetic acid and adding this solution to a solution of polyvinyl formal in N,N-dimethylformamide. (Polyvinyl formal, which is typically about 81% formalized, is soluble in N,N-dimethylformamide, as well as other solvents including tetrahydrofuran, dimethylsulfoxide and glacial acetic acid, among others.) This chitosan-polyvinyl formal solution is then added to polyanionic gelling solution such as an aqueous solution containing alginate to obtain gel particles.

For example, a process similar to that described in Pub. No. US 2003/0185895 to Lanphere et al. may be employed in which a polymeric solution containing a glucosamine polymer and a vinyl formal polymer (e.g., like that in the prior paragraph) is delivered to a drop generator. In certain embodiments, the polymeric solution may further contain and various additional agents (e.g., therapeutic agents, cell surface binding agents, imaging contrast agents, etc.). If desired, a viscosity controller may be used to heat the polymeric solution and reduce its viscosity prior to delivery to the drop generator. The drop generator forms and directs drops of the polymeric solution into a polyionic gelling solution (e.g., like that in the prior paragraph) resulting in ionically crosslinked particles.

In certain embodiments, gel-stabilized particles like those above may be transferred to a reactor vessel that includes an agent that chemically reacts with the polyol in the particles, thereby causing covalent crosslinking between or within the polymer chains. For instance, the vessel may include an aldehyde (e.g., formaldehyde or glutaraldehyde) and an acid, leading to acetalization of the hydroxyl groups in the polymers.

If not provided earlier in the process (e.g., by covalent bonding to one or more of the polymers used to form the particles or by admixture with the polymers during the particle forming process) various additional agents (e.g., therapeutic agents, cell surface binding agents, imaging contrast agents, etc.) may be covalently or non-covalently bound to the as-formed particles. For covalent or non-covalent bonding, the additional agent(s) may be exposed to the particles, for example, by coating the particles with, or soaking the particles in, a solution of the additional agent(s). Various processes are described above for covalent attachment of the additional agent(s) to the particles, including coupling via sulfonation and carbodiimide coupling. Non-covalent binding between the additional agent(s) and the particles may occurs spontaneously, based on the nature of the particle and the additional agent(s), for example, the respective charges of the particles and additional agent(s), and so forth.

In some embodiments, particles having basic groups (e.g., for instance, —NH₂ groups, etc.) may be admixed with an acidic agent (e.g., one having acidic groups such as —COOH groups, —SO₃H groups, —PO(OH)₂ groups, etc.). In other embodiments, particles having acidic groups may be admixed with a basic agent. In either case, acid-base neutralization may yield particles and agents of opposite charge, resulting in electrostatic interactions.

In some embodiments, salt forms of oppositely charged agents and particles are mixed. For example, particles having negatively charged groups (e.g., —COO⁻ groups,

—SO₃⁻ groups, —PO(OH)O⁻ groups, etc.) may be admixed with a positively charged agents (e.g., those having —NH₃⁺ groups, ═NH₂⁺ groups, etc.), or particles having positively charged groups may be admixed with negatively charged agents. Salt forms for positively charged particles/agents include those based on inorganic and organic acids (including amino acids, hydroxyacids and fatty acids), for instance, hydrochloride, hydrobromide, sulfate, nitrate, phosphate, mesylate, tosylate, acetate, propionate, maleate, benzoate, salicylate, fumarate, glutamate, aspartate, citrate, lactate, succinate, tartrate, hexanoate, octanoate, decanoate, oleate and stearate salt forms, among others. Salt forms for negatively charged particles/agents include those based on metals and amines (including amino acids), for instance, sodium, potassium, calcium, magnesium, zinc, triethylamine, ethanolamine, triethanolamine, meglumine, ethylene diamine, choline, arginine, lysine and histidine salt forms, among others.

The amount of each additional agent within the injectable particles of the present invention will vary widely, for example, with the amount of therapeutic agent being effective to treat the disease or disorder in question, with the amount of cell surface binding agent being effective to bind a sufficient fraction of the particles to tissue of interest, with the amount of imaging agent being effective to promote visualization, and so forth. Typical concentrations for the additional agents may range widely, for example, from about 0.1 wt % or less to 0.2 wt % to 0.5 wt % to 1 wt % to 2 wt % to 5 wt % to 10 wt % to 20 wt % to 25 wt % or more of the particle weight, among other possibilities.

The particles of the invention may be stored and transported in separate containers or in admixture. The particles of the invention may be stored and transported in dry form or in wet form (e.g., as an aqueous suspension). In addition to the agents discussed above, the particles of the invention may optionally contain additional agents such as one or more of the following among others: (a) tonicity adjusting agents including sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), (b) suspension agents including various surfactants and wetting agents, (c) imaging contrast agents (e.g., Omnipaque™, Visipaque™, etc.), and (d) pH adjusting agents including various buffer solutes. Dry or wet compositions may be shipped, for example, in a syringe, catheter, vial, ampoule, or other container. Dry forms may be mixed with an appropriate liquid carrier (e.g. sterile water for injection, physiological saline, phosphate buffer, a solution containing an imaging contrast agent, etc.) prior to administration. In this way the concentration of the particle composition to be injected may be varied at will, depending on the specific application at hand, as desired by the healthcare practitioner in charge of the procedure. Wet forms (e.g., aqueous suspensions) may also be mixed with a suitable liquid carrier (e.g. sterile water for injection, physiological saline, phosphate buffer, a solution containing contrast agent, etc.) prior to administration, allowing the concentration of administered particles (as well as other optional agents) in the suspension to be reduced prior to injection, if so desired by the healthcare practitioner in charge of the procedure. One or more containers of liquid carrier may also be supplied and shipped, along with the dry or wet particles, in the form of a kit. One or more containers of therapeutic agent, cell surface binding agent, or both, may also be supplied and shipped, along with the dry or wet particles, in the form of a kit.

In certain embodiments, the density of the liquid that suspends the particles is close to that of the particles themselves, thereby promoting an even suspension. The density of the aqueous phase may be increased, for example, by increasing the amount of solutes that are dissolved in the aqueous phase, and vice versa.

The effective amount of injectable particles within a suspension to be injected may be determined by those of ordinary skill in the art. An “effective amount” of any of the particles of the invention is, for example, (a) an amount sufficient to produce an occlusion or emboli at a desired site in the body, (b) an amount sufficient to achieve the degree of bulking desired (e.g., an amount sufficient to improve urinary incontinence, vesicourethral reflux, fecal incontinence, ISD or gastro-esophageal reflux, or an amount sufficient for aesthetic improvement), or (c) an amount sufficient to locally treat a disease, disorder or condition. Effective doses may also be extrapolated from dose-response curves derived from animal model test systems, among other techniques.

As noted above, permanent or temporary occlusion of blood vessels is useful for managing various diseases, disorders and conditions. Compositions including particles in accordance with the invention can thus be delivered to various sites in the body to effect permanent or temporary occlusion, including, for example, sites having tumors, such as those of the breast, prostate, lung, thyroid, or ovaries. The compositions can be used, for example, in neural, pulmonary, and/or AAA (abdominal aortic aneurysm) applications. The compositions can be used in the treatment of, for example, fibroids, tumors, internal bleeding, arteriovenous malformations (AVMs), and/or hypervascular tumors. The compositions can be used as, for example, fillers for aneurysm sacs, as fillers for AAA sacs (Type II endoleaks), as endoleak sealants, as arterial sealants, as puncture sealants, and can be used to provide occlusion of other lumens such as fallopian tubes. Internal bleeding includes gastrointestinal, urinary, renal and varicose bleeding, among other forms of bleeding. AVMs are, for example, abnormal collections of blood vessels (e.g. in the brain) which shunt blood from a high pressure artery to a low pressure vein, resulting in hypoxia and malnutrition of those regions from which the blood is diverted. In some embodiments, a composition containing the particles can be used to prophylactically treat a condition.

Fibroids, also known as leiomyoma, leiomyomata or fibromyoma, are the most common benign tumors of the uterus. These non-cancerous growths are present in significant fraction of women over the age of 35. In most cases, multiple fibroids are present, often up to 50 or more. Fibroids can grow, for example, within the uterine wall (“intramural” type), on the outside of he uterus (“subserosal” type), inside the uterine cavity (“submucosal” type), between the layers of broad ligament supporting the uterus (“interligamentous” type), attached to another organ (“parasitic” type), or on a mushroom-like stalk (“pedunculated” type). Fibroids may range widely in size, for example, from a few millimeters to 40 centimeters. In some women, fibroids can become enlarged and cause excessive bleeding and pain. While fibroids have been treated in the past by surgical removal of the fibroids (myomectomy) or by removal of the uterus (hysterectomy), recent advances in uterine embolization now offer a nonsurgical treatment. Injectable particles in accordance with the present invention can be used to treat uterine fibroids.

Methods for treatment of fibroids by embolization are well known to those skilled in the art (see, e.g., Pub. No. US 2003/0206864 to Mangin and the references cited therein). Uterine embolization is aimed at starving fibroids of nutrients. Numerous branches of the uterine artery may supply uterine fibroids. In the treatment of fibroids, embolization of the entire uterine arterial distribution network is often preferred. This is because it is difficult to selectively catheterize individual vessels supplying only fibroids, the major reason being that there are too many branches for catheterization and embolization to be performed in an efficient and timely manner. Also, it is difficult to tell whether any one vessel supplies fibroids rather than normal myometrium. In many women, the fibroids of the uterus are diffuse, and embolization of the entire uterine arterial distribution affords a global treatment for every fibroid in the uterus.

In a typical procedure, a catheter is inserted near the uterine artery by the physician (e.g., with the assistance of a guide wire). Once the catheter is in place, the guide wire is removed and contrast agent is injected into the uterine artery. The patient is then subjected to fluoroscopy or X-rays. In order to create an occlusion, an embolic agent is injected into the uterine artery via catheter. The embolic agent is carried by the blood flow in the uterine artery to the vessels that supply the fibroid. The particles flow into these vessels and clog them, thus disrupting the blood supply to the fibroid. In order for the physician to view and follow the occlusion process, contrast agent may be injected subsequent to infusion of the embolic agent.

Controlled, selective obliteration of the blood supply to tumors is also used in treating solid tumors such as renal carcinoma, bone tumor and liver cancer, among various others. The idea behind this treatment is that preferential blood flow toward a tumor will carry the embolization agent to the tumor thereby blocking the flow of blood which supplies nutrients to the tumor, thus, causing it to shrink. Embolization may be conducted as an enhancement to chemotherapy or radiation therapy.

Particles in accordance with the invention may also be used to treat various other diseases, conditions and disorders, including treatment of the following: arteriovenous fistulas and malformations including, for example, aneurysms such as neurovascular and aortic aneurysms, pulmonary artery pseudoaneurysms, intracerebral arteriovenous fistula, cavernous sinus dural arteriovenous fistula and arterioportal fistula, chronic venous insufficiency, varicocele, pelvic congestion syndrome, gastrointestinal bleeding, renal bleeding, urinary bleeding, varicose bleeding, uterine hemorrhage, and severe bleeding from the nose (epistaxis), as well as preoperative embolization (to reduce the amount of bleeding during a surgical procedure) and occlusion of saphenous vein side branches in a saphenous bypass graft procedure, among other uses.

Particles in accordance with the invention may also be used in tissue bulking applications, for example, as augmentative materials in the treatment of urinary incontinence, vesicourethral reflux, fecal incontinence, intrinsic sphincter deficiency (ISD) or gastro-esophageal reflux disease, or as augmentative materials for aesthetic improvement. For instance, a common method for treating patients with urinary incontinence is via periurethral or transperineal injection of a bulking material. In this regard, methods of injecting bulking agents commonly require the placement of a needle at a treatment region, for example, periurethrally or transperineally. The bulking agent is injected into a plurality of locations, assisted by visual aids, causing the urethral lining to coapt. In some cases, additional applications of bulking agent may be required.

The present invention encompasses various ways of administering the particulate compositions of the invention to effect embolization, bulking or other procedure benefiting the patient. One skilled in the art can determine the most desirable way of administering the particles depending on the type of treatment and the condition of the patient, among other factors. Methods of administration include, for example, percutaneous techniques as well as other effective routes of administration. For example, the particulate compositions of the invention may be delivered through a syringe or through a catheter, for instance, a FasTracker® microcatheter (Boston Scientific, Natick, Mass., USA), which can be advanced over a guidewire, a steerable microcatheter, or a flow-directed microcatheter (MAGIC, Balt, Montomorency, France).

Various aspects of the invention of the invention relating to the above are enumerated in the following paragraphs:

Aspect 1. Injectable particles comprising (a) a vinyl formal polymer, (b) a glucosamine polymer, and (c) an ionically or covalently bound agent selected from a therapeutic agent, a cell surface binding agent, and combinations thereof

Aspect 2. The injectable particles of aspect 1, wherein the glucosamine polymer comprises chitosan.

Aspect 3. The injectable particles of aspect 1, wherein the glucosamine polymer is derivatized with a saccharide moiety.

Aspect 4. The injectable particles of aspect 3, wherein the glucosamine polymer is galactosylated chitosan.

Aspect 5. The injectable particles of aspect 1, wherein the vinyl formal polymer is a copolymer comprising vinyl formal monomers and vinyl alcohol monomers.

Aspect 6. The injectable particles of aspect 1, wherein the vinyl formal polymer is a copolymer comprising vinyl formal monomers, vinyl alcohol monomers and vinyl acetate monomers.

Aspect 7. The injectable particles of aspect 1, wherein the vinyl formal polymer, the glucosamine polymer, or both, are derivatized with a polymeric moiety that has a positive or negative net charge at neutral pH.

Aspect 8. The injectable particles of aspect 1, wherein the vinyl formal polymer, the glucosamine polymer, or both are derivatized with a poly(amino acid) containing moiety that has a positive or negative net charge at neutral pH.

Aspect 9. The injectable particles of aspect 1, wherein the injectable particles further comprise a supplemental polymer that has a positive or negative net charge at neutral pH.

Aspect 10. The injectable particles of aspect 1, wherein the injectable particles comprise an ionically bound therapeutic agent.

Aspect 11. The injectable particles of aspect 1, wherein the injectable particles comprise a covalently bound therapeutic agent.

Aspect 12. The injectable particles of aspect 1, wherein the injectable particles comprise an ionically or covalently bound therapeutic agent selected from agents that kill cells, agents that slow or arrest cell growth, pain management agents, and combinations thereof.

Aspect 13. The injectable particles of aspect 1, wherein the injectable particles comprise an ionically bound cell surface binding agent.

Aspect 14. The injectable particles of aspect 1, wherein the injectable particles comprise a covalently bound cell surface binding agent.

Aspect 15. The injectable particles of aspect 1, wherein the injectable particles comprise an ionically or covalently bound cell surface binding agent selected from lectins, cell adhesion molecules (CAMs), extracellular matrix components, peptide binding sequences, antibodies for cell surface components, and combinations thereof.

Aspect 16. The injectable particles of aspect 1, wherein the particles comprise a covalently bound agent selected from therapeutic agents, cell surface binding agents, and combinations thereof.

Aspect 17. The injectable particles of aspect 16, wherein the covalently bound agent is coupled via amide linkages or via linkages formed using sulfonyl esters.

Aspect 18. The injectable particles of aspect 1, wherein the particles are ionically crosslinked.

Aspect 19. The injectable particles of aspect 1, wherein the particles are ionically crosslinked using a polyanionic crosslinking agent.

Aspect 20. The injectable particles of aspect 1, wherein the particles are covalently crosslinked.

Aspect 21. The injectable particles of aspect 1, wherein the particles are covalently crosslinked using an aldehyde.

Aspect 22. The injectable particles of aspect 1, wherein the particles are ionically and covalently crosslinked.

Aspect 23. The injectable particles of aspect 1, wherein the arithmetic mean maximum for the injectable particles is between 0.1 and 5 microns.

Aspect 24. The injectable particles of aspect 1, wherein the arithmetic mean maximum for the injectable particles is between 100 and 5000 microns.

Aspect 25. The injectable particles of aspect 1, wherein the first and second groups of polymeric particles have a sphericity of 0.8 or more.

Although various embodiments are specifically illustrated and described herein, it will be appreciated that modifications and variations of the present invention are covered by the above teachings and are within the purview of any appended claims without departing from the spirit and intended scope of the invention.

1. Injectable particles comprising (a) a vinyl formal polymer, (b) a glucosamine polymer, and (c) an ionically or covalently bound agent selected from a therapeutic agent, a cell surface binding agent, and combinations thereof.

2. The injectable particles of claim 1, wherein said glucosamine polymer comprises chitosan.

3. The injectable particles of claim 1, wherein said glucosamine polymer is derivatized with a saccharide moiety.

4. The injectable particles of claim 3, wherein said glucosamine polymer is galactosylated chitosan.

5. The injectable particles of claim 1, wherein said vinyl formal polymer is a copolymer comprising vinyl formal monomers and vinyl alcohol monomers.

6. The injectable particles of claim 1, wherein said vinyl formal polymer is a copolymer comprising vinyl formal monomers, vinyl alcohol monomers and vinyl acetate monomers.

7. The injectable particles of claim 1, wherein said vinyl formal polymer, said glucosamine polymer, or both, are derivatized with a polymeric moiety that has a positive or negative net charge at neutral pH.

8. The injectable particles of claim 1, wherein said vinyl formal polymer, said glucosamine polymer, or both are derivatized with a poly(amino acid) containing moiety that has a positive or negative net charge at neutral pH.

9. The injectable particles of claim 1, wherein said injectable particles further comprise a supplemental polymer that has a positive or negative net charge at neutral pH.

10. The injectable particles of claim 1, wherein said injectable particles comprise an ionically bound therapeutic agent.

11. The injectable particles of claim 1, wherein said injectable particles comprise a covalently bound therapeutic agent.

12. The injectable particles of claim 1, wherein said injectable particles comprise an ionically or covalently bound therapeutic agent selected from agents that kill cells, agents that slow or arrest cell growth, pain management agents, and combinations thereof.

13. The injectable particles of claim 1, wherein said injectable particles comprise an ionically bound cell surface binding agent.

14. The injectable particles of claim 1, wherein said injectable particles comprise a covalently bound cell surface binding agent.

15. The injectable particles of claim 1, wherein said injectable particles comprise an ionically or covalently bound cell surface binding agent selected from lectins, cell adhesion molecules (CAMs), extracellular matrix components, peptide binding sequences, antibodies for cell surface components, and combinations thereof.

16. The injectable particles of claim 1, wherein said particles comprise a covalently bound agent selected from therapeutic agents, cell surface binding agents, and combinations thereof.

17. The injectable particles of claim 16, wherein said covalently bound agent is coupled via amide linkages or via linkages formed using sulfonyl esters.

18. The injectable particles of claim 1, wherein said particles are ionically crosslinked.

19. The injectable particles of claim 1, wherein said particles are ionically crosslinked using a polyanionic crosslinking agent.

20. The injectable particles of claim 1, wherein said particles are covalently crosslinked.

21. The injectable particles of claim 1, wherein said particles are covalently crosslinked using an aldehyde.

22. The injectable particles of claim 1, wherein said particles are ionically and covalently crosslinked.

23. The injectable particles of claim 1, wherein said arithmetic mean maximum for the injectable particles is between 0. 1 and 5 microns.

24. The injectable particles of claim 1, wherein said arithmetic mean maximum for the injectable particles is between 100 and 5000 microns.

25. The injectable particles of claim 1, wherein said first and second groups of polymeric particles have a sphericity of 0.8 or more.
